(19) United States
(12) Patent Application Publication
(10) Pub. No.: US 2026/0000528 A1
(43) Pub. Date: Jan. 1, 2026

(54) Comfortable Toe Valgus Corrector

(71) Applicant: Hangzhou Moenmedic Health Technology Co., Ltd, Hangzhou (CN)

(72) Inventor: Junfei Jiang, Hangzhou (CN)

(21) Appl. No.: 18/899,631

(22) Filed: Sep. 27, 2024

(30) Foreign Application Priority Data
Jul. 1, 2024 (CN) 2024108721577
Jul. 1, 2024 (CN) 2024215450880

(51) Int. Cl. A61F 5/01

(52) U.S. Cl. CPC A61F 5/019

(57) ABSTRACT

A comfortable toe valgus corrector comprises a main support, a secondary support connected to the main support, and binding belts provided on the main support and the secondary support; the main support is connected to the secondary support via a connecting piece; wherein a supporting piece is provided below the main support, and the supporting piece can move up and down relative to the main support to change the distance between the two; a spring is provided between the supporting piece and the main support. The angle of the secondary support can be adjusted by the cooperation of the clamping grooves at different positions and the protrusions. By providing the spring, the supporting piece has a good buffer space, making it more comfortable for the user to wear the corrector, and the user will not feel uncomfortable even if the user wears it for a long time.

### TECHNICAL FIELD

The invention belongs to the technical field of toe correction, in particular to a comfortable toe valgus corrector.

### BACKGROUND ART

At present, due to people wearing shoes improperly or shoes that do not fit or other reasons, the toes may be deformed, turned inward, etc., especially when women wear high heels for a long time, the big toe of the foot may deviate toward the toe adjacent to the big toe, causing deformity of the big toe and affecting the beauty of the foot.

Although there are many correctors for the above problems, the existing correctors are not convenient and comfortable to use. For example, an easy-to-disassemble toe corrector is disclosed in the publication number CN214342832U and the publication date is Oct. 8, 2021, which discloses “an easy-to-disassemble toe corrector, comprising a main support, on which a main binding belt is provided for tying the main support to the arch of the foot; a secondary support, rotatably arranged on the main support; a secondary binding belt is provided on the secondary support for tying the secondary support to the big toe; a rotating column is provided on the main support, and an elastic clip is provided on the secondary support; the main support and the secondary support are connected by clamping the elastic clip on the rotating column. The invention uses the clamping and rotation form of the secondary support and the main support, so that the secondary support can be easily removed from the main support, thereby making it easy to replace the secondary support. When in use, the secondary support with different offset angles can be replaced to adapt to toes with different degrees of deformity.”

The above corrector needs to remove the secondary support to adjust the angle of the corrector, which is not convenient to use, and there is the trouble of losing multiple secondary supports. In addition, the existing ones directly use air bags to support the foot support points, and there is no good buffer structure such as springs. Users will feel stiff and poor fit during the correction process, resulting in poor comfort. Users may even feel pain after long-term use.

### SUMMARY OF THE INVENTION

The purpose of the invention is to provide a comfortable toe valgus corrector.

The invention is achieved as follows:

A comfortable toe valgus corrector, comprising a main support, a secondary support connected to the main support, and binding belts provided on the main support and the secondary support; the main support is connected to the secondary support via a connecting piece; wherein a supporting piece is provided below the main support, and the supporting piece can move up and down relative to the main support to change the distance between the two; a spring is provided between the supporting piece and the main support.

As a further improvement, a center of the connecting piece is provided with a through-going slide rail; the slide rail is movably connected to a moving rod, and the moving rod can move up and down in the slide rail.

As a further improvement, the center of the connecting piece is provided with a threaded hole, and the threaded hole is cooperated with an adjusting piece with a thread on the top; an interior of the adjusting piece is a hollow structure, and the interior of the adjusting piece is movably connected to the moving rod; the moving rod is columnar and is fixed to a center of the supporting piece by screws.

As a further improvement, a top of the moving rod is provided with a centrally symmetrical and outwardly extending fan-shaped limiting block; the shape of the limiting block is matched with the shape of the slide rail at the center of the adjusting piece to ensure that the moving rod can move up and down along the slide rail and can also drive the adjusting piece to rotate through the moving rod; the spring is arranged on the moving rod between the connecting piece and the supporting piece.

As a further improvement, the spring is a conical spring with increasing perimeter from top to bottom; a top of the spring contacts the adjusting piece, and a bottom of the spring is arranged inside a spring groove provided on the supporting piece; a bottom of the adjusting piece is provided with a limiting part to prevent the moving rod from falling downwards.

As a further improvement, the main support comprises a first main support and a second main support; the first main support and the second main support are connected to the connecting piece via an annular flange extending outward from a bottom of the connecting piece, and the annular flange is arranged in a limiting groove formed by the combination of the first main support and the second main support; the main support can rotate horizontally relative to the secondary support connected to the connecting piece above via the connecting piece.

As a further improvement, the secondary support can rotate relative to the connecting piece.

As a further improvement, a top of the connecting piece is hemispherical; both front and rear of the connecting piece are symmetrically provided with a plurality of clamping grooves arranged vertically and horizontally, and both left and right sides of the connecting piece are provided with a connecting hole; the connecting hole is movably connected to a connecting column provided on the secondary support to achieve longitudinal rotation of the secondary support; one end of the secondary support is provided with a hemispherical ball cover, and an inside of the ball cover is provided with protrusions that are cooperated with the clamping grooves; when the secondary support is rotated to a position parallel to the main support, the highest point of a bottom surface on the secondary support is in the same horizontal plane as a circular bottom surface of the main support.

As a further improvement, square steel rings are fixed symmetrically between the first main support and the second main support; the steel ring is connected to the binding belt, and ends of the binding belts are attached to each other via Velcro; one side of the second main support is provided with a support pad, and the first main support is provided with a curved groove with left-right mirror symmetry.

As a further improvement, the secondary support is provided with through slots, and the binding belts are connected to the through slots; the support piece is provided with a silicone sleeve; the secondary support is provided with an arc-shaped bottom surface.

The following are the outstanding and beneficial technical effects of the invention compared with the prior art.
(1) A plurality of clamping grooves arranged vertically are provided on the connecting piece, one end of the secondary support is provided with a hemispherical ball cover, and an inside of the ball cover is provided with protrusions that are cooperated with the clamping grooves. The angle of the secondary support can be adjusted by the cooperation of the clamping grooves at different positions and the protrusions.
(2) A spring is provided between the supporting piece and the main support. When the user wears the corrector, the spring is deformed to apply an inward force to the side of the user's big toe; at the same time, the secondary support applies an outward rotational force to the big toe. Due to the presence of the spring, the corrector has a better correction effect. In addition, by providing the spring, the supporting piece has a good buffer space, making it more comfortable for the user to wear the corrector, and the user will not feel uncomfortable even if the user wears it for a long time.
(3) The moving rod can move up and down along the slide rail and can also drive the adjusting piece to rotate through the moving rod. The user can adjust the distance between the supporting piece and the main support by rotating the supporting piece, thereby meeting the correction needs of different users.
(4) The design of the spring of the corrector allows the supporting piece to fit the foot better when the user is wearing the corrector and walking; in addition, the user can use the expansion and contraction of the spring to repeatedly pull and correct the toes while walking.
(5) The use of the conical spring can compress to a greater extent than other springs, thereby providing a larger adjustment space and greater elastic force. The use of the conical spring and the provision of a spring groove at the bottom of the conical spring can ensure that the structure in contact with and connected to the spring is more compact and stable, allowing the supporting piece to better fit the user's foot.
(6) The main support can rotate 360 degrees horizontally relative to the secondary support connected to the connecting piece above via the connecting piece. The rotation angle is large and the structure is more flexible. The main support and the secondary support can be rotated horizontally at will while the user is walking, so that the user can feel more comfortable while walking after wearing the corrector.

### BRIEF DESCRIPTION OF ACCOMPANY DRAWINGS

FIG. 1 is a schematic diagram of the split structure according to the invention.

FIG. 2 is a schematic diagram of the overall structure according to the invention.

FIG. 3 is a schematic structural diagram of the secondary support according to the invention.

FIG. 4 is a schematic structural diagram of the moving rod according to the invention.

FIG. 5 is a schematic diagram of a partially enlarged structure according to the invention.

FIG. 6 is a partial cross-sectional view of the moving rod according to the invention.

FIG. 7 is a schematic structural diagram of another embodiment of the connecting piece according to the invention.

### SPECIFIC EMBODIMENT OF THE INVENTION

The invention will be further described hereinafter with reference to the embodiments.

### Embodiment 1

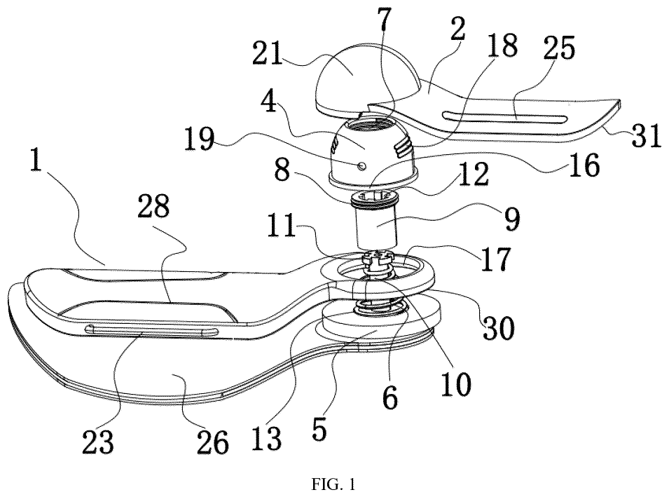

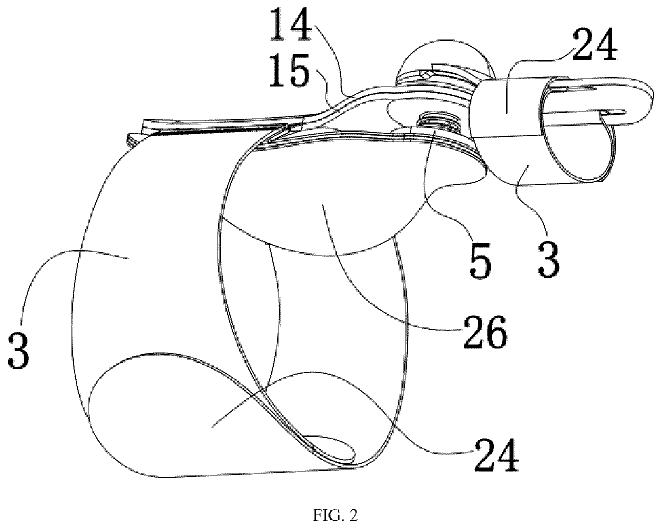

As shown in FIGS. 1 and 2, a comfortable toe valgus corrector, comprising a main support 1 located on the inner side of the user's foot when in use and a secondary support 2 connected to the main support 1. After the user puts it on, the secondary support 2 is located on the side of the user's big toe. It also comprises binding belts 3 provided on the main support 1 and the secondary support 2, and the binding belts 3 are used to fix the main support 1 and the secondary support 2 to the user's feet; the main support 1 is connected to the secondary support 2 via a connecting piece 4. A supporting piece 5 is provided below the main support 1. After the user puts it on, the supporting piece 5 is located on the side of the user's foot, slightly below the big toe of the foot, and the supporting piece 5 is used as a fulcrum to rotate the inward-deflected big toe outward through the secondary support to achieve correction of the big toe position. In order to make the toe valgus corrector suitable for people with different degrees of toe deformation, the supporting piece 5 is arranged to be movable up and down relative to the main support 1 to change the distance between the two. In addition, in order to improve wearing comfort and enhance user experience, a spring 6 is provided between the supporting piece 5 and the main support 1; the spring 6 can achieve a very good cushioning effect. Compared with the existing toe valgus corrector without the spring 6, the user can feel that the supporting piece 5 is not so stiff and uncomfortable after wearing it, and the user will not feel uncomfortable even wearing it for a long time. When the user uses the corrector, the supporting piece 5 will be subjected to very large pressure when in contact with the foot, so it is necessary to provide a spring 6 with a buffering function on the supporting piece 5.

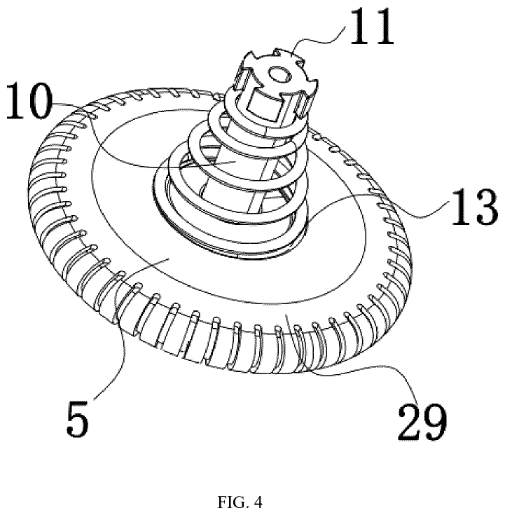

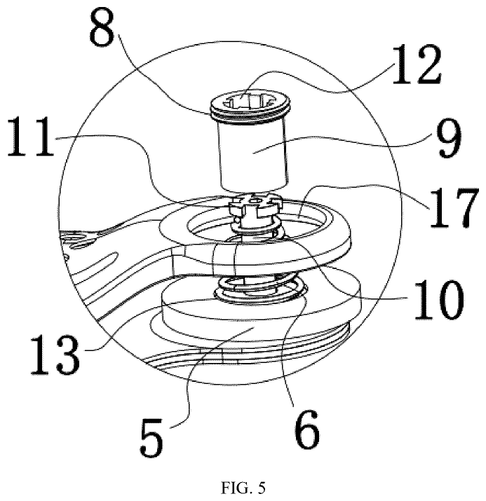

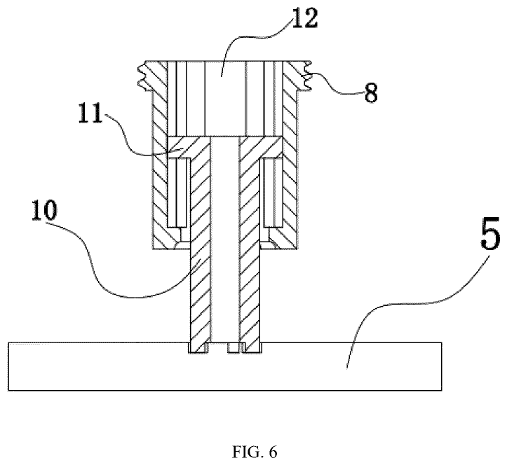

As shown in FIGS. 1, 4, 5, and 6, the center of the connecting piece 4 is provided with a threaded hole 7, and the threaded hole 7 is cooperated with an adjusting piece 9 with a thread 8 on the top, so that the adjusting piece 9 can move up and down inside the connecting piece 4. An interior of the adjusting piece 9 is a hollow structure, and the interior of the adjusting piece 9 is movably connected to the moving rod 10; the moving rod 10 can also move up and down inside the adjusting piece 9. The moving rod 10 is columnar and is fixed to a center of the supporting piece 5 by screws; a top of the moving rod 10 is provided with a centrally symmetrical and outwardly extending fan-shaped limiting block 11; the shape of the limiting block 11 is matched with the shape of the slide rail 12 at the center of the adjusting piece 9 to ensure that the moving rod 10 can move up and down along the slide rail 12 and can also drive the adjusting piece 9 to rotate through the moving rod 10. When the user needs to adjust the distance between the supporting piece 5 and the main support 1, the supporting piece 5 can drive the moving rod 10 fixedly connected to the supporting piece 5 to rotate, and the moving rod 10 can drive the adjusting piece 9 to rotate through the limiting block 11 during the rotation process. During the rotation of the adjusting piece 9, the thread 8 at the top thereof moves up and down inside the connecting piece 4, thereby finally achieving the adjustment of the distance between the supporting piece 5 and the main support 1; the user can adjust the distance between the above two according to their own acceptance level. The spring 6 is arranged on the moving rod 10 between the connecting piece 9 and the supporting piece 5. When the supporting piece 5 is subjected to force, it will move toward the inside of the adjusting piece 9 through the moving rod 10. When the supporting piece 5 is not subjected to force, it will return to its original state downward under the restoring force of the spring 6.

The spring 6 is a conical spring with increasing perimeter from top to bottom; a top of the spring 6 contacts the adjusting piece 9, and a bottom of the spring 6 is arranged inside a spring groove 13 provided on the supporting piece 5. The use of a conical spring and arranging a spring groove 13 at the bottom of the spring 6 can ensure that the structure in contact with and connected to the spring 6 is more compact and stable, and can also make the supporting piece 5 fit the user's foot better. A bottom of the adjusting piece 9 is provided with a limiting part to prevent the moving rod 10 from falling downwards, and the limiting part can prevent the moving rod 10 from falling downwards by simply blocking the slide rail 12.

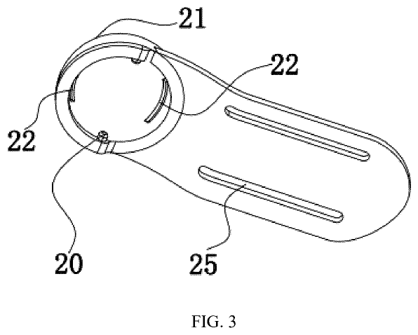

As shown in FIGS. 2 and 3, the main support 1 comprises a first main support 14 and a second main support 15; the first main support 14 and the second main support 15 are connected to the connecting piece 4 via an annular flange 16 extending outward from a bottom of the connecting piece 4, and the annular flange 16 is arranged in a limiting groove 17 formed by the combination of the first main support 14 and the second main support 15. The above connection method does not completely fix the main support 1 and the connecting piece 4, but allows the main support 1 to rotate relative to the connecting piece 4. The movable connection structure is more flexible and changeable, which is convenient for different users.

In addition, the secondary support 2 can rotate relative to the connecting piece 4, which is achieved specifically through the following structure: a top of the connecting piece 4 is hemispherical; both front and rear of the connecting piece 4 are symmetrically provided with a plurality of clamping grooves 18 arranged vertically and horizontally, and both left and right sides of the connecting piece 4 are provided with a connecting hole 19; the connecting hole 19 is movably connected to a connecting column 20 provided on the secondary support 2 to achieve longitudinal rotation of the secondary support 2; one end of the secondary support 2 is provided with a hemispherical ball cover 21, and an inside of the ball cover 21 is provided with protrusions 22 that are cooperated with the clamping grooves 18. In actual use, the user only needs to move the secondary support 2 by hand to make the protrusions 22 snap into the clamping grooves 18 at different positions, thereby realizing the change of the angle of the secondary support 2 relative to the main support 1, so as to adapt to people with different degrees of toe deformation and make more appropriate correction. Compared with the existing secondary support 2 with different angles that can be detached and replaced, it is more convenient. When the secondary support 2 is rotated to a position parallel to the main support 1, the highest point of a bottom surface 31 on the secondary support 2 is in the same horizontal plane as a circular bottom surface 30 of the main support 1. Due to the presence of the spring 6, the height of the circular bottom surface 30 of the secondary support 2 is lowered relative to the existing secondary support so that it can better fit the user's feet, and the optimal effect is achieved when the height is lowered to be on the same level as the circular bottom surface 30 of the main support 1.

As shown in FIGS. 1 and 2, square steel rings 23 are fixed symmetrically between the first main support 14 and the second main support 15; the steel ring 23 is connected to the binding belt 3, and ends of the binding belts 3 are attached to each other via Velcro 24. The secondary support 2 is provided with through slots 25, and the binding belts 3 are connected to the through slots 25. The user can easily and quickly wear the corrector through the Velcro on the binding belts 3. One side of the second main support 15 is provided with a support pad 26, and the support pad 26 can make the user more comfortable after wearing; the first main support 14 is provided with a curved groove 28 with left-right mirror symmetry, and the curved groove 28 makes the product more beautiful and lighter. The secondary support 2 is provided with an arc-shaped bottom surface 31, which makes the user more comfortable to wear.

### Embodiment 2

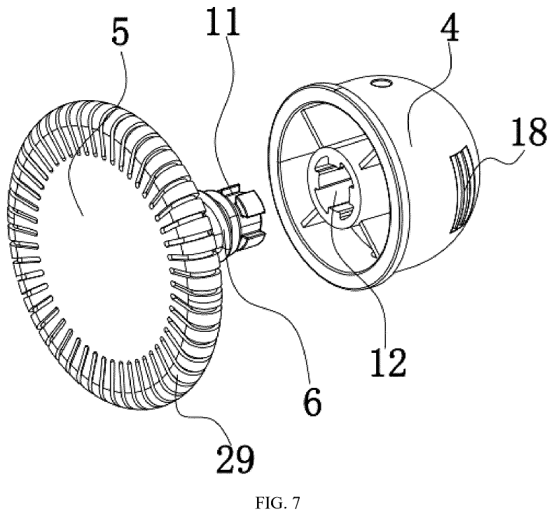

As shown in FIG. 7, compared with Embodiment 1, the adjusting piece 9 in Embodiment 1 is directly fixed inside the connecting piece 4 through reinforcing ribs and the threaded connection between the two is cancelled; at this time, the two can be regarded as a whole, and only the slide rail 12 is provided in a middle of the connecting piece 4 to be cooperated with the limiting block 11, and the function of adjusting by rotation in Embodiment 1 is cancelled, making the structure simpler and more stable.

The above embodiments are only preferred embodiments of the invention and are not intended to limit the protection scope of the invention. Therefore, any equivalent changes made based on the structure, shape, and principle of the invention shall all fall within the protection scope of the invention.

1. A comfortable toe valgus corrector, comprising a main support, a secondary support connected to the main support, and binding belts provided on the main support and the secondary support; the main support is connected to the secondary support via a connecting piece; wherein a supporting piece is provided below the main support, and the supporting piece can move up and down relative to the main support to change the distance between the two; a spring is provided between the supporting piece and the main support.

2. The comfortable toe valgus corrector of claim 1, wherein a center of the connecting piece is provided with a through-going slide rail; the slide rail is movably connected to a moving rod, and the moving rod can move up and down in the slide rail.

3. The comfortable toe valgus corrector of claim 1, wherein the center of the connecting piece is provided with a threaded hole, and the threaded hole is cooperated with an adjusting piece with a thread on the top; an interior of the adjusting piece is a hollow structure, and the interior of the adjusting piece is movably connected to the moving rod.

4. The comfortable toe valgus corrector of claim 3, wherein the moving rod is columnar and is fixed to a center of the supporting piece by screws; a top of the moving rod is provided with a centrally symmetrical and outwardly extending fan-shaped limiting block; the shape of the limiting block is matched with the shape of the slide rail at the center of the adjusting piece to ensure that the moving rod can move up and down along the slide rail and can also drive the adjusting piece to rotate through the moving rod; the spring is arranged on the moving rod between the connecting piece and the supporting piece.

5. The comfortable toe valgus corrector of claim 4, wherein the spring is a conical spring with increasing perimeter from top to bottom; a top of the spring contacts the adjusting piece, and a bottom of the spring is arranged inside a spring groove provided on the supporting piece; a bottom of the adjusting piece is provided with a limiting part to prevent the moving rod from falling downwards.

6. The comfortable toe valgus corrector of claim 1, wherein the main support comprises a first main support and a second main support; the first main support and the second main support are connected to the connecting piece via an annular flange extending outward from a bottom of the connecting piece, and the annular flange is arranged in a limiting groove formed by the combination of the first main support and the second main support; the main support can rotate horizontally relative to the secondary support connected to the connecting piece above via the connecting piece.

7. The comfortable toe valgus corrector of claim 1, wherein the secondary support can rotate relative to the connecting piece.

8. The comfortable toe valgus corrector of claim 7, wherein a top of the connecting piece is hemispherical; both front and rear of the connecting piece are symmetrically provided with a plurality of clamping grooves arranged vertically and horizontally, and both left and right sides of the connecting piece are provided with a connecting hole; the connecting hole is movably connected to a connecting column provided on the secondary support to achieve longitudinal rotation of the secondary support; one end of the secondary support is provided with a hemispherical ball cover, and an inside of the ball cover is provided with protrusions that are cooperated with the clamping grooves; when the secondary support is rotated to a position parallel to the main support, the highest point of a bottom surface on the secondary support is in the same horizontal plane as a circular bottom surface of the main support.

9. The comfortable toe valgus corrector of claim 6, wherein square steel rings are fixed symmetrically between the first main support and the second main support; the steel ring is connected to the binding belt, and ends of the binding belts are attached to each other via Velcro; one side of the second main support is provided with a support pad, and the first main support is provided with a curved groove with left-right mirror symmetry.

10. The comfortable toe valgus corrector of claim 1, wherein the secondary support is provided with through slots, and the binding belts are connected to the through slots; the support piece is provided with a silicone sleeve; the secondary support is provided with an arc-shaped bottom surface.
